(19) United States
(12) Patent Application Publication
(10) Pub. No.: US 2017/0311927 A1
(43) Pub. Date: Nov. 2, 2017

(54) APPARATUS

(71) Applicant: CANON KABUSHIKI KAISHA, Tokyo (JP)

(72) Inventors: Reiko Yao, Yokohama-shi (JP); Ryuichi Nanaumi, Tokyo (JP)

(21) Appl. No.: 15/493,268

(22) Filed: Apr. 21, 2017

(30) Foreign Application Priority Data
Apr. 28, 2016 (JP) 2016-090681

(51) Int. Cl. A61B 8/00; G01S 7/52; A61B 8/08; G01S 15/89

(52) U.S. Cl. CPC A61B 8/4466; G01S 15/8993; G01S 7/52028; A61B 8/483; A61B 8/08; A61B 8/44; G01S 15/89

(57) ABSTRACT

An apparatus that acquires information on an object includes an element that converts an acoustic wave propagating from the object through a holding member into a reception signal at an element position and an information processor that uses the reception signal to generate characteristic information on the object. The information processor determines, for each unit region of the object, whether the unit region is a unit region for numerical analysis in which the delay time of the acoustic wave is acquired by numerical analysis, or a unit region for interpolation in which the delay time is acquired by interpolation processing; performs numerical analysis on the unit region for numerical analysis.

### BACKGROUND OF THE INVENTION

### Field of the Invention

The present invention relates to an apparatus.

### Description of the Related Art

Techniques for acquiring characteristic information on an object such as a living body by receiving and analyzing an acoustic wave propagated from the object have been developed in the medical field and the like. For example, there are photoacoustic apparatuses for determining optical characteristics of an object on the basis of a photoacoustic wave generated when the object is irradiated with light.

In some cases, a member, through which an acoustic wave is propagated at a velocity of sound different from the velocity of sound in the object, is disposed between the object and an acoustic wave receiver in such an object information acquiring apparatus, Examples of such a member include a holding member for holding the object and an acoustic matching material for matching the acoustic impedances of the object and the acoustic wave receiver. In such cases, there is a technique for calculating the propagation path of the acoustic wave by Snell's law, calculating the delay time from the propagation path, and correcting the influence of refraction (Japanese Patent Application Laid-open No. 2010-167258).

Patent Literature 1: Japanese Patent Application Laid-open No. 2010-167258

### SUMMARY OF THE INVENTION

However, in order to calculate the delay time, it is necessary to use a numerical analysis such as a bisection method which is an iterative method. Since numerical analysis accompanying iterative processing requires processing of a plurality of sound rays, the amount of calculation becomes very large. As a result, the processing time may be prolonged, or the scale and cost of the apparatus may be increased, which may cause problems in terms of practicality. In particular, where the unit region (pixel or voxel) is refined to improve the resolution, or the number of elements in the receiver is increased, the amount of calculation is greatly increased.

The present invention has been created in view of the above problems. An objective of the present invention is to speed up processing by reducing the amount of calculation in an apparatus that acquires information on an object by using acoustic waves.

The present invention provides an apparatus configured to acquire characteristic information in a plurality of unit regions in an object by using a signal obtained by receiving an acoustic wave generated from the object with a receiver, the apparatus comprising:

a first acquirer configured to acquire information on a delay time corresponding to part of unit regions among the plurality of unit regions by using information on a velocity of sound in a propagation path of the acoustic wave from the unit regions to the receiver;

a second acquirer configured to acquire information on a delay time corresponding to the remaining unit regions among the plurality of unit regions by interpolation processing using the information on the delay time corresponding to the part of the unit regions; and

a third acquirer configured to acquire the characteristic information by using the information on the delay time corresponding to part of unit regions or the information on a delay time corresponding to the remaining unit regions to determine a signal corresponding to the delay time for each of the plurality of the unit regions, and using the determined signal.

The present invention also provides an apparatus configured to generate characteristic information on an object by using a reception signal obtained by conversion with an element from an acoustic wave that propagates from the object through a holding member and is incident on the element at an element position, wherein

determination is made, for each unit region which has been set in the object, whether the unit region is a unit region for numerical analysis in which a delay time of the acoustic wave incident from the unit region on the element position is acquired by numerical analysis, or a unit region for interpolation in which the delay time is acquired by interpolation processing;

with respect to the unit region for numerical analysis, the delay time is acquired by numerical analysis using a path of the acoustic wave and a velocity of sound in the object and in the holding member;

in the unit region for interpolation, the delay time is acquired using the delay time acquired with respect to the unit region for numerical analysis, and the reception signal is selected for each unit region from a memory on the basis of the delay time; and

the characteristic information is generated using the reception signal.

According to the present invention, it is possible to speed up processing by reducing the amount of calculation in an apparatus that acquires information on an object by using acoustic waves.

Further features of the present invention will become apparent from the following description of exemplary embodiments with reference to the attached drawings.

### BRIEF DESCRIPTION OF THE DRAWINGS

FIGS. 1A and 1B are functional block diagrams of an object information acquiring apparatus in Example 1;

FIGS. 2A and 2B are flowcharts showing the processing flow in Example 1;

FIG. 3 is a diagram showing a numerical analysis target or an interpolation target in Example 1;

FIG. 4 is a flowchart showing the delay time calculation processing flow in Example 1;

FIGS. 5A and 5B are diagrams showing a processing group and a subgroup in Example 1;

FIG. 6 is a functional block diagram of an information processor in Example 2;

FIGS. 7A and 7B are flowcharts showing the processing flow in Example 2;

FIG. 8 is a flowchart showing a delay time calculation processing flow in Example 2; and

FIG. 9 is a schematic diagram relating to setting of a wave type.

### DESCRIPTION OF THE EMBODIMENTS

The preferred embodiments of the present invention will be described below with reference to the drawings. However, the dimensions, materials, shapes, of the components described below, relative positions thereof, and the like, should be appropriately changed according to the configuration of the apparatus to which the invention is applied and various conditions. Therefore, the scope of the present invention is not intended to be limited to the following description.

The present invention relates to a technique for detecting acoustic waves propagating from an object, generating characteristic information on the inside of the object, and acquiring the characteristic information. Therefore, the present invention can be understood as relating to an object information acquiring apparatus or a control method thereof, or an object information acquiring method or a signal processing method. The present invention can be also understood as relating to a program for causing an information processing apparatus equipped with hardware resources such as a CPU and a memory to execute these methods, a storage medium that stores the program, or an information processing apparatus.

The object information acquiring apparatus of the present invention is inclusive of an apparatus that uses a photoacoustic effect to receive acoustic waves generated in an object when the object is irradiated with light (electromagnetic waves) and to acquire characteristic information on the object as image data. In this case, the characteristic information is information on characteristic values corresponding to each of a plurality of positions in the object and is generated using a reception signal obtained by receiving a photoacoustic wave.

The characteristic information acquired by photoacoustic measurements is a value reflecting the absorption rate of light energy. For example, it may include a source of an acoustic wave generated by light irradiation, an initial sound pressure in the object, an optical energy absorbing density or an absorbing coefficient derived from the initial sound pressure, and the concentration of the substance constituting the tissue. Further, an oxygen saturation degree distribution can be calculated by determining the oxygenated hemoglobin concentration and reduced hemoglobin concentration as the substance concentration. In addition, for instance, glucose concentration, collagen concentration, melanin concentration, and volume fraction of fat and water also can be determined.

The object information acquiring apparatus of the present invention is inclusive of an apparatus using an ultrasonic echo technique by which an ultrasound wave is transmitted to an object, a reflected wave (echo wave) reflected inside the object is received, and object information is acquired as image data. In this case, the object information which is to be acquired is information reflecting the difference in the acoustic impedance of the tissue inside the object.

Two- or three-dimensional characteristic information distribution can be obtained on the basis of characteristic information at each position in the object. Distribution data can be generated as image data. The characteristic information may be also obtained as distribution information at each position in the object, rather than numerical data. Thus, the distribution information can be the initial sound pressure distribution, energy absorbing density distribution, absorbing coefficient distribution, or oxygen saturation degree distribution.

The acoustic wave as referred to in the present invention is typically an ultrasound wave and includes elastic waves called sound waves and acoustic waves. An electrical signal obtained by conversion from an acoustic wave with a probe or the like is also called an acoustic signal. However, the terms “ultrasound waves” and “acoustic waves” in this description are not intended to limit the wavelength of these elastic waves. An acoustic wave generated by a photoacoustic effect is called a photoacoustic wave or a light-induced ultrasound wave. An electrical signal derived from the photoacoustic wave is also called a photoacoustic signal. An electrical signal derived from an ultrasonic echo is also called an ultrasound wave signal.

### EXAMPLE 1

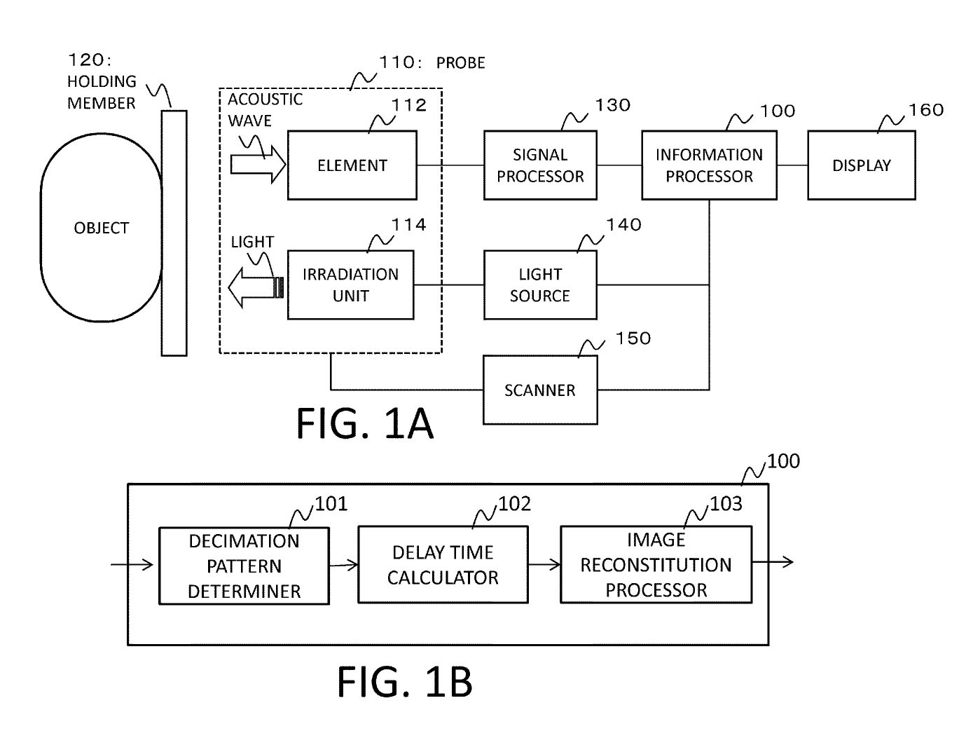

FIG. 1 is a functional block diagram showing the configuration of the object information acquiring apparatus according to Example 1. The overall configuration will be explained with reference to FIG. 1A. The object information acquiring apparatus in this example is a photoacoustic apparatus and includes an information processor 100, a probe 110, a holding member 120, a signal processor 130, a light source 140, a scanner 150, and a display 160. The probe 110 includes an irradiation unit 114 for irradiating an object (for example, a part of a living body such as a breast) with light propagated from the light source 140 by an optical system, and an element 112 for receiving a photoacoustic wave generated by the photoacoustic effect from a light absorber inside the object and converting the received wave into an electrical signal.

FIG. 1B shows details of the information processor 100. The information processor 100 performs image reconstruction for each unit region (pixel or voxel) which has been set in the region of interest of the object to acquire information indicating optical characteristics, and generates image data showing a characteristic information distribution inside the object. Thus, the region of interest constitutes the entire calculation region from which the characteristic information is acquired. The information processor includes a decimation pattern determiner 101, a delay time calculator 102, and an image reconstruction processor 103. In the following example, pixels are assumed as unit regions. However, processing related to voxels in the case of three-dimensional image reconstruction can also be executed in the same manner.

The decimation pattern determiner 101 determines a pattern of pixels (pixels for numerical analysis) for calculating the characteristic information values by numerical analysis and pixels (pixels for interpolation) for calculation by approximation by interpolation. In the present example, a plurality of line-shaped pixel groups is selected at equal intervals in a two-dimensional region. For example, various other patterns can be taken, such as decimation of every other pixel, decimation of every few pixels, etc. The pattern determined here is the initial setting. Depending on the wave type reaching an element from each pixel, there are cases where numerical analysis is performed also on pixels which have been set as pixels for interpolation.

The delay time calculator 102 calculates the delay time to be applied to each pixel. Here, the delay time refers to the time required for an acoustic wave generated in a certain pixel to reach a certain element position. The delay time is calculated on the basis of the type of the medium located in the propagation path of the acoustic wave (for example, the object, the holding member, the acoustic matching material, etc.) and the velocity of sound in each medium. For that purpose, the delay time calculator 102 specifies the wave type based on the positional relationship (in particular, angle) between each pixel and each element, and calculates the delay time from surrounding pixels of the same wave type by using interpolation. Even when the relative position of the probe and the object is changed by scanning, the delay time can be applied to a plurality of elements according to the relationship between the pixel and the element position.

In the present invention, the delay time calculator is a first acquirer that acquires information on a delay time corresponding to unit regions which are part of a plurality of unit regions and are for numerical analysis. Further, the delay time calculator also combines the functions of the first acquirer and a second acquirer that acquires a delay time corresponding to the remaining unit regions (unit regions for interpolation) of the plurality of unit regions by interpolation processing using information on the delay time corresponding to part of the unit regions (unit regions for numerical analysis).

The image reconstruction processor 103 performs image reconstruction processing using the delay time calculated by the delay time calculator 102. The image reconstruction processor corresponds to the third acquirer of the present invention.

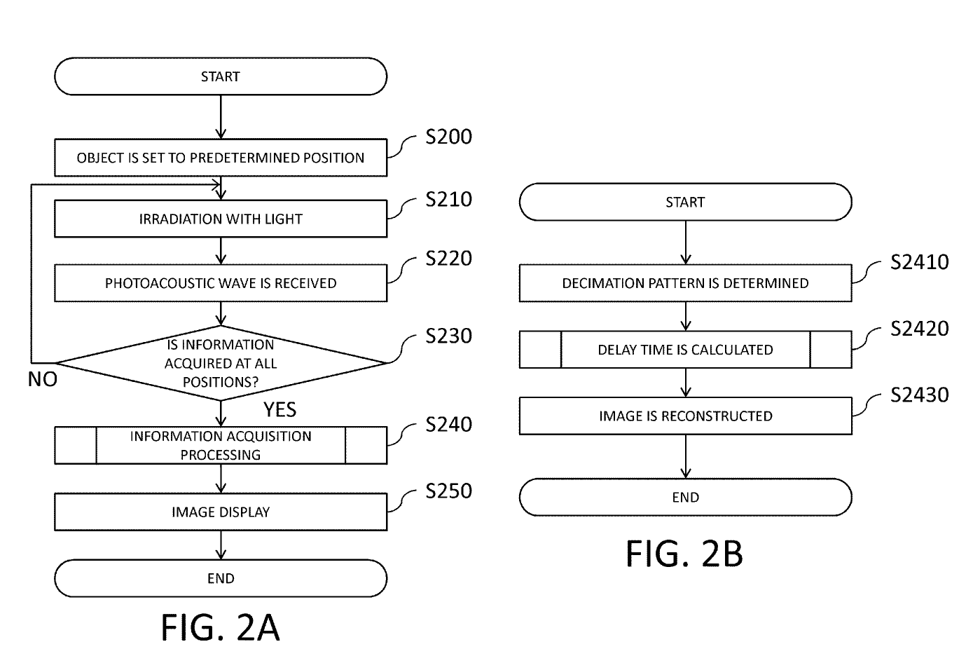

Next, a basic flow will be described with reference to FIG. 2. FIG. 2A is an overall flow for acquiring object information. In step S200, a technician sets the object at a predetermined position. For example, a breast is housed in a cup-shaped holding member 120. In step S210, the object is irradiated with light from the light source 140. In step S220, each element 112 receives a photoacoustic wave generated from the object, converts it into electrical signals, and outputs the electrical signals. The electrical signals are sequentially stored in the memory (storage means) of the information processor 100 after being digitalized and amplified by the signal processor 130.

In the above-described processing, the steps S210 and S220 are continued (step S230) until the acquisition of data in the predetermined region of interest is completed by the scanning of the probe 110. In step S240, information acquisition processing to be described in detail later is performed, and image data indicating characteristic information inside the object are generated. In step S250, an image based on the image data is displayed on a display 160.

Step S2410 in FIG. 2B is a step of determining a decimation interval. The decimation pattern determiner 101 of the present example selects pixels for numerical analysis from pixels included in the region of interest. The pixels for numerical analysis, as referred to herein, are pixels for which a delay time is obtained by calculation based on a propagation path from the pixels to a target element, the degree of refraction or reflection between members, velocity of sound in each member, and the like. This corresponds to “part of unit regions” or “unit regions for numerical analysis” of the present invention. Meanwhile, other pixels are the pixels for interpolation. This corresponds to the “remaining unit regions” or “unit regions for interpolation” of the present invention. A general value may be used as the velocity of sound within each member, or an actually measured value may be used.

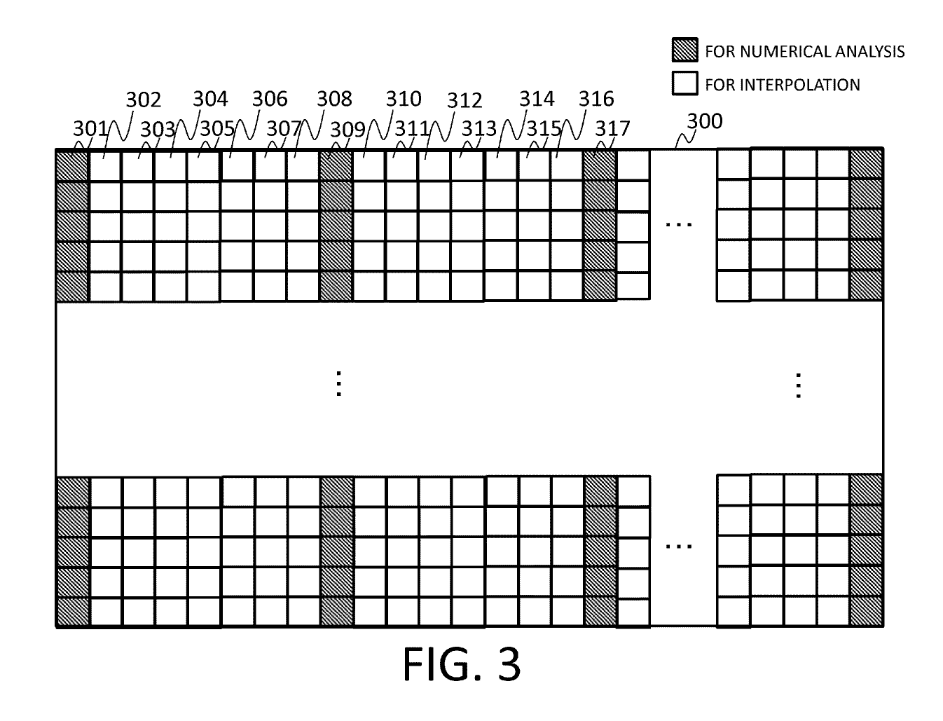

FIG. 3 is a pattern showing whether each pixel of one image 300 in the present example is a pixel for interpolation or a pixel for numerical analysis. Pixel groups corresponding to reference numerals 301, 309, and 317 in FIG. 3 are for numerical analysis. Meanwhile, pixel groups denoted by reference numerals 302 to 308 and 310 to 316 are for interpolation. In the present example, line-shaped pixel groups to be calculated by numerical analysis without interpolation are set at equal intervals (every eight pixels).

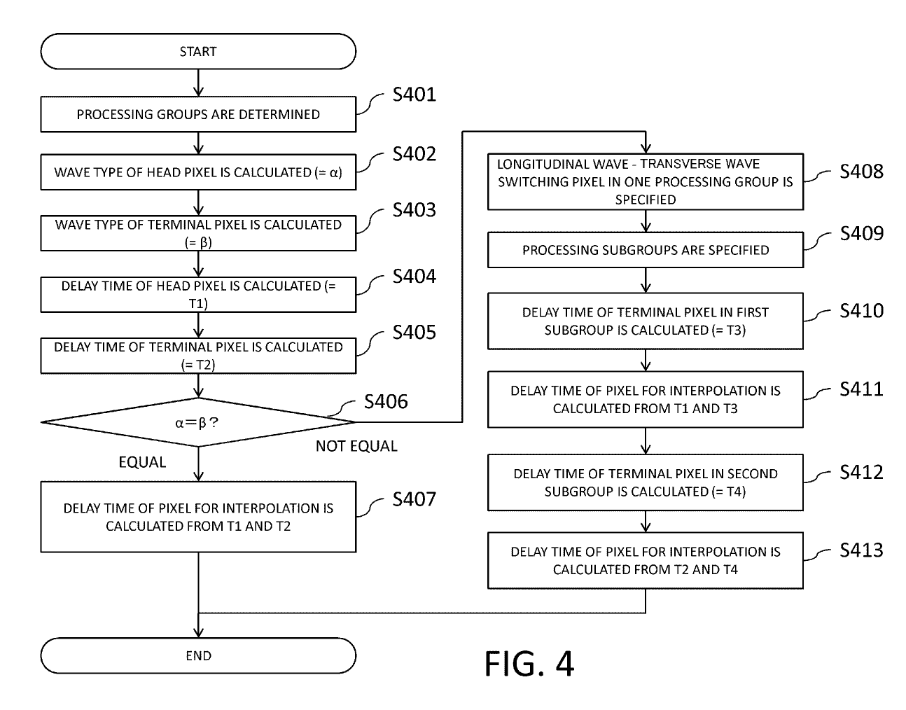

Returning to the description of the processing flow, in step S2420, the delay time calculator 102 calculates the delay time for each pixel at each element position. A specific process implemented in step S2420 will be explained with reference to FIG. 4.

FIG. 4 is a flowchart showing the detailed process of delay time calculation in step S2420 in FIG. 2. In step S401, pixels are divided into processing groups by a divider. Specifically, a group of pixels between a pixel (pixel for numerical analysis) which is to be calculated by numerical analysis without interpolation and a next pixel for numerical analysis present in the lateral direction on the paper sheet is taken as one processing group. The divider for performing this processing may be included together with, for instance, the decimation pattern determiner 101 in the information processor 100, or the functions thereof may be realized by the decimation pattern determiner 101 or the delay time calculator 102. Alternatively, when the information processor 100 acquires a predetermined processing group which has been determined in advance and stored in a memory or the like, the functions of the divider may be realized by a processing module therefor.

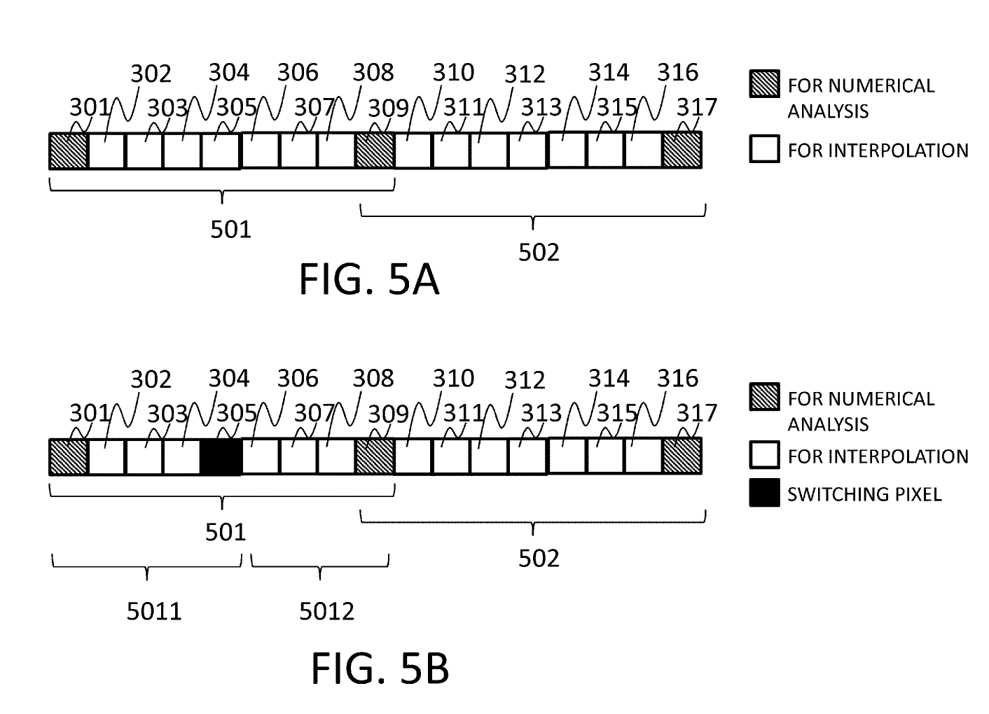

An example of the processing group division in S401 will be explained using FIG. 5A. For convenience, only the uppermost row of pixels in FIG. 3 is shown. Actually, the same processing is performed for each row. As described hereinabove, in the present example, since the pixels for numerical analysis are arranged for each predetermined number (here, eight pixels), in the range depicted the figure, there are three reference numerals 301, 309, and 317. Therefore, the pixel group within the range of reference numerals of 301 to 309 is determined as a processing group 501, and the pixel group within the range of reference numerals of 309 to 317 is determined as a processing group 502. Here, the number of cycles of numerical analysis can be reduced by including one pixel for numerical analysis in a plurality of processing groups, as shown by the reference numeral 309. In the figure, the second and subsequent rows are omitted. However, these pixels can be similarly divided into processing groups for each predetermined number (here, nine).

Returning to the description of FIG. 4, in step S402, the wave type of the head pixel of the group divided in step S401 is calculated and determined by the determiner. The wave type is a longitudinal wave or a transverse wave. The wave type calculated in step S402 is defined as a. The wave type can be determined according to the angle of incidence formed by each pixel and the probe. However, other methods may be used to set the wave type. For example, when processing of the group 501 is performed in the order from the left side on the page, it is determined whether a is a longitudinal wave or a transverse wave according to the angle at which the acoustic wave is incident on the element 112 (or element position), which is the examination target, from the pixel 301. The determiner for performing this processing may be included together with, for instance, the decimation pattern determiner 101 in the information processor 100, or the functions thereof may be realized by the decimation pattern determiner 101 or the delay time calculator 102.

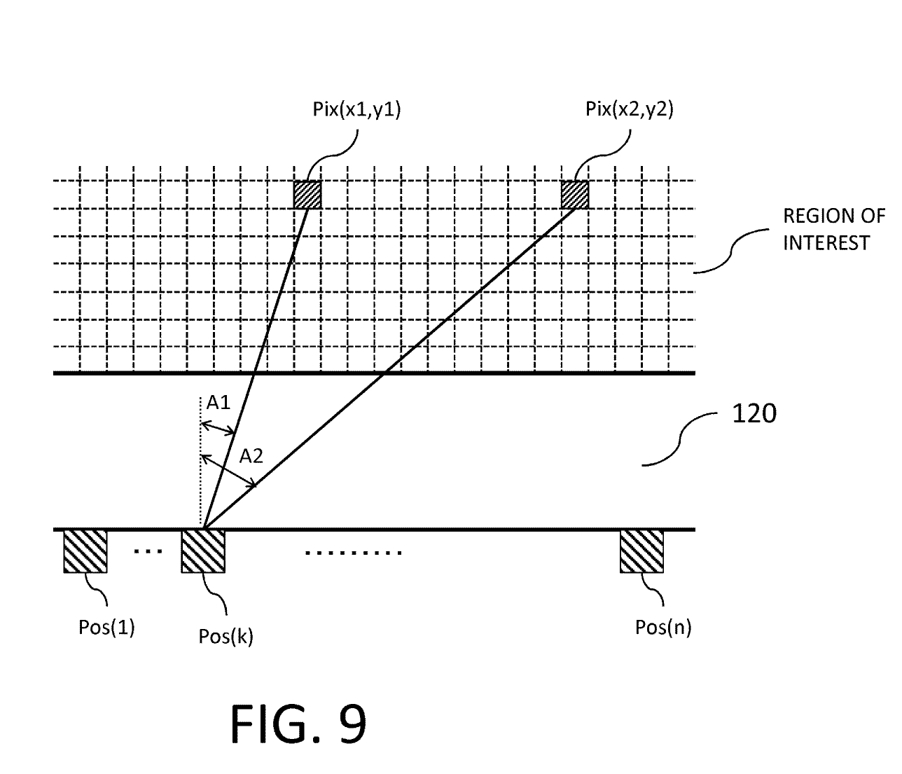

In particular, when the holding member is a solid body, the acoustic wave propagating from the object via the holding member shows different reflection and refraction characteristics depending on the angle of incidence due to the difference in the critical angle between the longitudinal wave and the transverse wave. Typically, when the angle of incidence of an acoustic wave on the holding member is at least the critical angle, the wave type is taken as the transverse wave. Simplifying, when the angle of incidence of an acoustic wave on an element position is at least a predetermined angle determined according to the critical angle, the wave type may be taken as a transverse wave. FIG. 9 is a schematic diagram exemplifying this case. An acoustic wave propagated from each pixel in the region of interest is incident on the element positions Pos (l) to Pos (n) through the holding member 120. Here, considering Pos (k), since the angle A1 of incidence from the pixel Pix (x1, y1) on the element position Pos (k) is less than the predetermined angle, the longitudinal wave is set. Meanwhile, since the angle A2 of incidence from the pixel Pix (x2, y2) on the element position Pos (k) is larger than the predetermined angle, the transverse wave is set.

In step S403, the wave type of the terminal pixel of the group determined in S401 is calculated. The wave type calculated in step S403 is defined as β. In this case, the wave type can be determined by the same method as in S402.

In step S404, the delay time (T1) of the head pixel is calculated. The head delay time T1 is a value calculated by numerical analysis without interpolation. T1 indicates the time from the head pixel to a certain element. Therefore, when a multi-probe is used, T1 differs for each element. This also applies to other delay times. The delay time can be calculated based on the path (and path length) of the acoustic wave which represents the refraction or reflection state, and the velocity of sound in the object, holding member, acoustic matching material, or the like.

In step S405, the delay time (T2) of the terminal pixel is calculated. The terminal delay time T2 is also a value calculated by numerical analysis. In step S406, it is determined whether or not the wave type a calculated in step S402 matches the wave type β calculated in step S403. When it is determined that α and β match, the processing advances to step S407. When it is determined that α and β do not match, the processing advances to step S408.

In step S407, a delay time to be applied to pixels for interpolation other than the head and terminal pixels in the processing group is calculated from the head delay time T1 calculated in step S404 and the terminal delay time T2 calculated in step S405. An arbitrary method such as a linear interpolation method, a bilinear method, a bicubic method, or the like, can be used as a calculation method, according to the capability of the information processing apparatus, required image accuracy, and the like.

Step S408 corresponds to a case where pixels having different wave types are present in the processing group determined in step S401. In this step, the pixel for which the wave type is to be switched is specified within the processing group determined in step S401. The wave type can be specified in the same manner as in steps S402 and S403.

In step S409, pixel groups included in the same wave type are determined as subgroups on the basis of the pixel information in which the wave type is switched. FIG. 5B shows an example of processing subgroup determination. In the figure, it is assumed that the wave type switching pixel for which the longitudinal wave and transverse wave are switched, this pixel being specified in step S408, has the reference numeral 305. Here, it is assumed that the pixels 301 to 305 are determined to be of the wave type a and the pixels 306 to 309 are determined to be of the wave type β. In this case, the pixels 301 to 305 are determined to be of the same wave type group and are determined as a subgroup 5011. Meanwhile, the pixels 306 to 309 are determined as a subgroup 5012. Hereinafter, they are referred to as the first subgroup 5011 and the second subgroup 5012, respectively, and distinguished from each other. The way to divide the subgroups varies depending on the location of the pixel for which the wave type is switched. The number of pixels in each subgroup also changes.

Returning to the description of FIG. 4, in step S410, the delay time (T3) of the terminal pixel in the first subgroup 5011 is calculated. The delay time T3 is a value calculated by numerical analysis without interpolation. Thus, with respect to the reference numeral 305 initially set as the pixel for interpolation, numerical analysis is performed by the processing of the present step, thereby improving the accuracy of interpolation processing.

In step S411, delay times for pixels other than the head and terminal pixels in the first subgroup are calculated by approximation by interpolation from the delay time T1 of the head pixel of the processing group and the delay time T3 of the terminal pixel of the first subgroup 5011. Thus, the delay times of the pixels 302 to 304 are acquired. As in the above, any interpolation method can be used.

In step S412, the delay time of the head pixel in the second subgroup 5012 is calculated. Thus, the delay time (T4) of the pixel 306 is calculated. The second subgroup head pixel delay time T4 is a value calculated by numerical analysis without interpolation.

In step S413, interpolation processing is performed using the delay time T2 calculated in step S405 (also the second subgroup terminal pixel delay time) and the second subgroup head pixel delay time T4. Thus, the delay times for pixels other than the head and terminal pixels in the second subgroup are calculated by approximation by interpolation. In this example, the delay times of the pixels 307 and 308 are calculated. As in the above, a desired interpolation method can be used. It should be noted that a method for selecting the pixels for numerical analysis and the range of pixels for interpolation for which the interpolation processing is to be performed in one cycle are not limited to those described above. For example, pixels for numerical analysis may be selected in a checkered pattern or the like, and the interpolation may be performed for a predetermined range (for example, 10×10 pixels).

Returning to FIG. 2, in step S2430, the image reconstruction processor 103 performs image reconstruction. Any method such as a phasing addition method, a filtered back projection method, a Fourier transform method, and an inverse computation method can be used for image reconstruction.

According to the present example, it is possible to specify the wave type of a pixel and to calculate the delay time by interpolation from surrounding pixels of the same wave type. Here, the delay time used for reading out the reception signal from the memory at the time of reconstruction is a value acquired by calculation for the pixels for numerical analysis, and a value acquired in the interpolation process for the pixels for interpolation. Therefore, it is possible to select an appropriate reception signal in the reconstruction while accelerating the processing. As a result, high-precision images can be acquired at a high speed.

### PREFERRED EXAMPLE

The preferred example of each block of the object information acquiring apparatus will be described hereinbelow. As an object to be measured according to the present invention, a part of a living body such as a breast is assumed. Phantoms for calibration and non-destructive inspection targets can be also measured.

The information processor 100 can be realized by an information processing apparatus such as a PC or workstation, which includes a CPU, a memory, a communication device, an input unit (user interface), and the like. Each block such as the decimation pattern determiner 101, the delay time calculator 102, and the image reconstruction processor 103 in the information processor 100 can be realized as modules of a program which is stored in a memory in the information processing apparatus and operated using computational resources of the image processing apparatus.

Various elements that receive an acoustic wave, convert the received wave into an electrical signal, and output the electrical signal can be used as the element 112. For example, a piezoelectric element, a cMUT, a Fabry-Perot probe, or the like, can be used. Where the object information acquiring apparatus is an ultrasound echo device, the element 112 may transmit the ultrasound wave to the object, or a transmitting element may be provided separately from the receiving element. The signal processor 130 includes an AD conversion circuit for digitizing an analog electrical signal, an amplifier circuit, and the like. These circuits can be realized by a processing circuit configured of an FPGA, an ASIC, or the like.

A pulse laser device capable of obtaining a large output is preferable as the light source 140. The wavelength of the laser light is preferably in the near-infrared range. It is preferable to use light having a wavelength with a high absorbing coefficient by the absorber which is the measurement target. Further, by using light having a plurality of wavelengths, it is possible to acquire substance concentration related information such as oxygen saturation degree. In addition to laser devices, flash lamps and LEDs can also be used. The laser light outputted from the light source 140 is conducted by an optical system such as an optical fiber, a mirror, a lens, or the like, and is radiated from the irradiation unit 114.

A probe in which a plurality of elements is arranged one-dimensionally or two-dimensionally is preferable as the probe 110. The resulting effect is that the measurement time can be shortened and the SN ratio can be increased. Where a hemispherical or bowl-shaped member is used as the probe 110, it is possible to form a high-sensitivity region in which directions (directional axes) with high reception sensitivity of the respective elements 112 are concentrated. As a result, an image with good contrast can be generated. In that case, a cup-shaped member may be used as the holding member 120. A handheld casing may be used as the probe 110.

For example, an acrylic resin, polymethylpentene, polyethylene terephthalate, or the like, having high transparency to light or acoustic waves can be used as the holding member 120. Further, it is preferable to dispose an acoustic matching material for matching the acoustic impedance between the holding member 120 and the object. Water, castor oil, ultrasonic gel, and the like, are suitable as the acoustic matching material. Further, when there is a space between the holding member 120 and the element 112, it is preferable to dispose the acoustic matching material therein. The point of calculating the propagation path and the delay time of the acoustic wave with consideration for the velocity of sound in each member remains unchanged even when an acoustic matching material is used.

The scanner 150 changes the relative position of the object and the probe. By using the scanner 150, it is possible to measure a wide area of the object. An XY stage equipped with a positioning mechanism or a power mechanism can be used as the scanner 150. The display 160 displays an image based on image data generated by image reconstruction. The display 160 may be provided separately from the object information acquiring apparatus.

### EXAMPLE 2

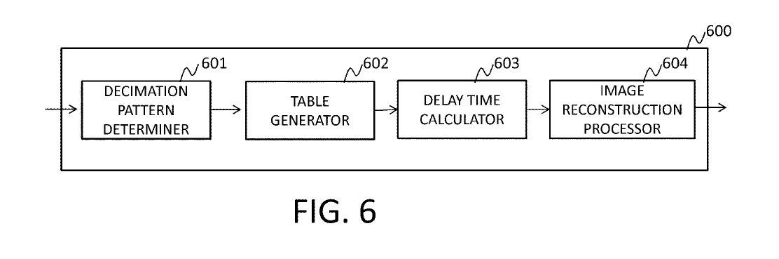

In the explanation of Example 2 hereinbelow, the attention is focused on the difference from Example 1. FIG. 6 is a functional block diagram showing the configuration of the information processor 600 of the present example. In FIG. 6, functions and configurations of a decimation pattern determiner 601 and an image reconstruction processor 604 are the same as those in Example 1.

A table generator 602 generates a table in which the longitudinal wave is used to calculate the delay time and a table in which the transverse wave is used to calculate the delay time with respect to pixels to be calculated by numerical analysis without interpolation. The table in which the longitudinal wave is used to calculate the delay time is defined hereinbelow as a longitudinal wave table, and a table in which the transverse wave is used to calculate the delay time is defined as a transverse wave table. A delay time calculator 603 of the present example calculates the delay time by using the longitudinal wave table and the transverse wave table generated by the table generator 602.

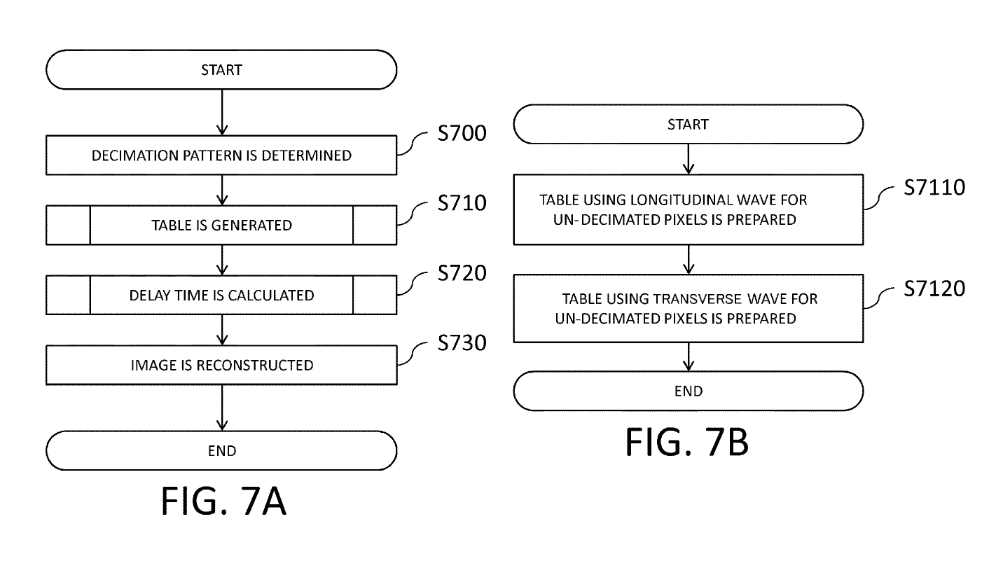

In the explanation of the processing flow of the present example hereinbelow, the attention is focused on the difference from Example 1. In FIG. 7A, the processing starts from the point of time at which the photoacoustic wave is received from the object and information acquisition processing is performed. In step S700, the decimation pattern determiner 601 determines a decimation interval. In the present example, the pixels for interpolation and the pixels for numerical analysis are selected with the pattern such as shown in FIG. 3.

In step S802, the table generator 602 generates a table. Details of this processing will be described with reference to FIG. 7B. In step S7110, a table in which the longitudinal wave is used to calculate the delay time, that is, the longitudinal wave table, is generated for the pixels (pixels for numerical analysis) for calculating the delay time by numerical analysis without interpolation. The pixels for numerical analysis correspond to the pixels 301, 309, and 317 in FIG. 3. In the following step S7120, a table in which the transverse wave is used to calculate the delay time, that is, the transverse wave table, is generated for the pixels (pixels for interpolation) for which the delay time is calculated by interpolation. The pixels for interpolation correspond to the pixels 302 to 308 and 310 to 316 in FIG. 3.

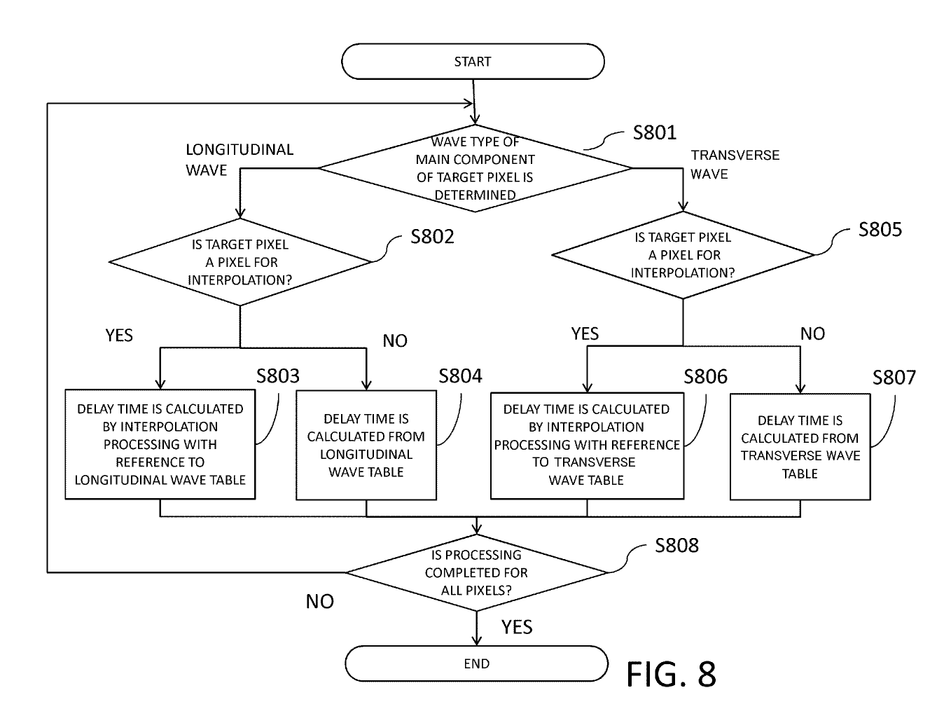

Returning to FIG. 7A, in step S703, the delay time calculator 603 calculates the delay time. Specific processing performed at this time will be described with reference to FIG. 8. In step S801, the wave type of the main component of the target pixel of the processing included in the region of interest is determined. Where the wave type is determined to be a longitudinal wave, the processing advances to step S802, and where the wave type is determined to be a transverse wave, the processing advances to step S805. The specification of the wave type can be determined, for example, according to the angle of incidence formed between each pixel and the probe. Where the longitudinal wave component and the transverse wave component are included in a certain pixel, the main component indicates the component included in a larger amount.

In step S802, it is determined whether or not the target pixel for which the longitudinal wave is the main component wave type is the pixel for interpolation. Where the target pixel is for interpolation, the processing advances to step S803, and where the target pixel is not for interpolation, the processing advances to step S804. In step S803, the delay time of the target pixel is calculated by interpolation processing on the basis of the longitudinal wave table values of pixels surrounding the target pixel with reference to the longitudinal wave table generated by the table generator 702 in S7110 of FIG. 7B. For interpolation, any method such as linear interpolation can be used. In step S804, the longitudinal wave table value corresponding to the target pixel is acquired by referring to the longitudinal wave table value, and the acquired value is taken as the delay time.

In step S805, it is determined whether or not the target pixel for which the transverse wave is the main component wave type is the pixel for interpolation. Where the target pixel is for interpolation, the processing advances to step S806, and where the target pixel is not for interpolation, the processing advances to step S807. In step S806, the delay time of the target pixel is calculated by interpolation processing on the basis of the transverse wave table values of pixels surrounding the target pixel with reference to the transverse wave table generated by the table generator 702 in S7120 of FIG. 7B. For interpolation, any method such as linear interpolation can be used. In step S807, the transverse wave table value corresponding to the target pixel is acquired by referring to the transverse wave table value, and the acquired value is taken as the delay time.

Returning to FIG. 7A, in step S730, image reconstruction is executed in the same manner as in step S2430 of FIG. 2B. According to the sequence described above, a table generated according to the wave type of the longitudinal wave and the transverse wave is generated and applied to each pixel according to the main component wave type of the target pixel, and the delay time is calculated. As a result, high-speed processing can be realized.

### VARIATION EXAMPLE

In the description hereinabove, the photoacoustic wave generated from the object by the photoacoustic effect was considered. The present invention is also applicable to echo waves in which ultrasound waves transmitted from each element to the object are reflected by a change in acoustic impedance in the object.

The present invention is not limited only to the apparatus and method for realizing the abovementioned embodiments. For example, the present invention supplies a program code of software for realizing the abovementioned embodiments in a computer (CPU or MPU) in the system or apparatus. The case where the abovementioned embodiments are realized as a result of the computer of the system or the apparatus causing the various devices to operate according to the program code is also included in the scope of the present invention.

Also, in this case, the program code itself of the software realizes the functions of the abovementioned embodiments. Therefore, the program code itself and a means for supplying the program code to the computer, more specifically, a storage medium storing the program code, are included in the scope of the present invention. For example, a floppy disk, a hard disk, an optical disk, a magneto-optical disk, a CD-ROM, a DVD, a magnetic tape, a nonvolatile memory card, a ROM, or the like, can be used as a storage medium for storing such program code. The storage medium may be a computer-readable non-transitory storage medium that stores the program.

Further, the computer controls various devices only according to the supplied program code. Not only the case in which the functions of the abovementioned embodiments are thus realized, but also the case in which the abovementioned embodiments are realized in cooperation with an OS (operating system) on which the program code is running on a computer, or other application software is also included in the scope of the present invention.

Further, after the supplied program code has been stored in the memory provided in a function expansion board or function storage unit, the CPU provided in the function expansion board or function storage unit performs the entire actual processing or part thereof on the basis of the instruction of the program code. The case where the abovementioned embodiments are realized by such processing is also included in the scope of the present invention.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-090681, filed on Apr. 28, 2016, which is hereby incorporated by reference herein in its entirety.

What is claimed is:

1. An apparatus configured to acquire characteristic information in a plurality of unit regions in an object by using a signal obtained by receiving an acoustic wave generated from the object with a receiver, the apparatus comprising:
a first acquirer configured to acquire information on a delay time corresponding to part of unit regions among the plurality of unit regions by using information on a velocity of sound in a propagation path of the acoustic wave from the unit regions to the receiver;
a second acquirer configured to acquire information on a delay time corresponding to the remaining unit regions among the plurality of unit regions by interpolation processing using the information on the delay time corresponding to the part of the unit regions; and
a third acquirer configured to acquire the characteristic information by using the information on the delay time corresponding to part of unit regions or the information on a delay time corresponding to the remaining unit regions to determine a signal corresponding to the delay time for each of the plurality of the unit regions, and using the determined signal.

2. The apparatus according to claim 1, further comprising
a divider configured to divide the plurality of unit regions, which have been set in the object, into processing groups for every predetermined number, wherein
the delay time corresponding to the remaining unit regions is acquired by interpolation processing based on the delay time corresponding to the part of unit regions included in the processing group.

3. The apparatus according to claim 2, further comprising
a determiner configured to determine a wave type of the acoustic wave incident on a position of the receiver from the unit region, for each of the unit regions included in the processing group, wherein
when the delay time corresponding to the remaining unit regions is acquired, interpolation processing is performed using the delay time of the part of the unit regions having the same wave type.

4. The apparatus according to claim 3, wherein the determiner is configured to determine the wave type in accordance to an angle of incidence of the acoustic wave incident on the position of the receiver from the unit region.

5. The apparatus according to claim 4, wherein
the wave type is either a longitudinal wave or a transverse wave.

6. The apparatus according to claim 5, wherein
the determiner is configured to determine the wave type as a transverse wave when the angle of incidence is at least a predetermined angle determined according to a critical angle of the acoustic wave.

7. The apparatus according to claim 3, wherein
the second acquirer is configured to divide the unit region included in the processing group into a plurality of subgroups according to the wave type and performs the interpolation processing for each of the subgroups.

8. The apparatus according to claim 1, further comprising:
a light source configured to irradiate the object with light, wherein
the acoustic wave is a photoacoustic wave generated from the object.

9. The apparatus according to claim 1, wherein
the acoustic wave is an echo wave transmitted from the receiver and reflected by the object.

10. The apparatus according to claim 1, wherein
the plurality of unit regions constitute an entire calculation region in which the characteristic information is acquired.

11. The apparatus according to claim 1, further comprising:
the receiver, and
a memory configured to store a signal outputted from the receiver.

12. The apparatus according to claim 1, comprising:
a holder configured to hold the object.

13. An apparatus configured to generate characteristic information on an object by using a reception signal obtained by conversion with an element from an acoustic wave that propagates from the object through a holding member and is incident on the element at an element position, wherein
determination is made, for each unit region which has been set in the object, whether the unit region is a unit region for numerical analysis in which a delay time of the acoustic wave incident from the unit region on the element position is acquired by numerical analysis, or a unit region for interpolation in which the delay time is acquired by interpolation processing;
with respect to the unit region for numerical analysis, the delay time is acquired by numerical analysis using a path of the acoustic wave and a velocity of sound in the object and in the holding member;
in the unit region for interpolation, the delay time is acquired using the delay time acquired with respect to the unit region for numerical analysis, and
the reception signal is selected for each unit region from a memory on the basis of the delay time; and
the characteristic information is generated using the reception signal.
